(19) United States
(12) Patent Application Publication
(10) Pub. No.: US 2015/0329546 A1
(43) Pub. Date: Nov. 19, 2015

(54) NOVEL CRYSTALLINE FORM OF TICAGRELOR AND PROCESS FOR THE PREPARATION THEREOF

(71) Applicant: ACTAVIS GROUP PTC EHF, Hafnarfjordur (IS)

(72) Inventors: Krishnadatt SHARMA, Mumbai (IN); Nandkumar GAIKWAD, Navi Mumbai (IN); Nikhil TRIVEDI, Navi Mumbai (IN); Nitin Sharadchandra PRADHAN, Thane (West) (IN)

(21) Appl. No.: 14/360,719

(22) PCT Filed: Nov. 29, 2012
(86) PCT No.: PCT/EP12/73951

§ 371 Date: May 27, 2014

(30) Foreign Application Priority Data
Nov. 30, 2011 (IN) 4143/CHE/2011

(51) Int. Cl. C07D 487/04

(52) U.S. Cl. CPC C07D 487/04; C07B 2200/13

(57) ABSTRACT

The present invention refers to a new crystalline form of ticagrelor, a process for the preparation thereof, pharmaceutical compositions comprising said new crystalline form of ticagrelor, and the use of the new crystalline form of ticagrelor as medicament

### FIELD OF THE INVENTION

The present invention refers to a new crystalline form of ticagrelor, a process for the preparation thereof, pharmaceutical compositions comprising said new crystalline form of ticagrelor, and the use of the new crystalline form of ticagrelor as medicament.

### BACKGROUND OF THE INVENTION

U.S. Pat. Nos. 6,251,910 and 6,525,060 disclose a variety of triazolo[4,5-d]pyrimidine derivatives, processes for their preparation, pharmaceutical compositions comprising the derivatives, and method of use thereof. These compounds act as P_{2T} (P2Y_{ADP} or P2T_{AC}) receptor antagonists and they are indicated for use in therapy as inhibitors of platelet activation, aggregation and degranulation, promoters of platelet disaggregation and anti-thrombotic agents. Among them, Ticagrelor, [1S-(1α,2α,3β(1 S*,2R*),5β)]-3-[7-[2-(3,4-difluorophenyl)cyclopropyl]amino]-5-(propylthio)-3H-1,2,3-triazolo[4,5-d]pyrimidin-3-yl)-5-(2-hydroxyethoxy)-cyclopentane-1,2-diol, acts as Adenosine uptake inhibitor, Platelet aggregation inhibitor, P2Y12 purinoceptor antagonist and Coagulation inhibitor. It is indicated for the treatment of thrombosis, angina, Ischemic heart diseases and coronary artery diseases. Ticagrelor is represented by the following structural formula I:

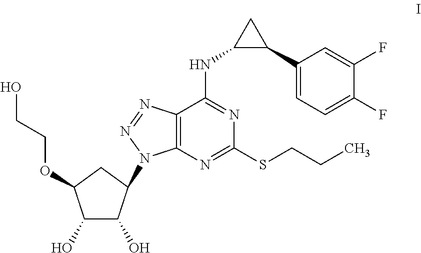 [SMILES: CCCSc1nc(N[C@@H]2C[C@H]2c2ccc(F)c(F)c2)c2nnn([C@@H]3C[C@H](OCCO)[C@@H](O)[C@H]3O)c2n1]

Ticagrelor is the first reversibly binding oral adenosine diphosphate (ADP) receptor antagonist and is chemically distinct from thienopyridine compounds like clopidogrel. It selectively inhibits P2Y12, a key target receptor for ADP. ADP receptor blockade inhibits the action of platelets in the blood, reducing recurrent thrombotic events. The drug has shown a statistically significant primary efficacy against the widely prescribed clopidogrel (Plavix) in the prevention of cardiovascular (CV) events including myocardial infarction (heart attacks), stroke, and cardiovascular death in patients with ACS.

Various processes for the preparation of ticagrelor, its enantiomers and related compounds, and their pharmaceutically acceptable salts are disclosed in U.S. Pat. Nos. 6,251,910; 6,525,060; 6,974,868; 7,067,663; and 7,250,419; U.S. Patent application Nos. 2007/0265282, 2007/0293513 and 2008/0214812; and European Patent Nos. EP0996621 and EP1135391; and PCT Publication Nos. WO2008/018823 and WO2010/030224.

According to U.S. Pat. No. 5,747,496, the 4,6-dichloro-5-nitro-2-(propylthio)pyrimidine of formula II is prepared by adding propyl iodide to a suspension of 4,6-dihydroxy-2-mercaptopyrimidine in water containing sodium hydroxide; the reaction mixture is stirred for 2 weeks and then the reaction mass is concentrated to half volume; followed by the addition of hydrochloric acid and isolating the product by filtration to produce 2-propylthio-pyrimidine-4,6-diol. The 2-propylthio-pyrimidine-4,6-diol is then reacted with excess fuming nitric acid to produce 5-nitro-2-propylthiopyrimidine-4,6-diol. The 5-nitro-2-propylthiopyrimidine-4,6-diol is reacted with phosphoryl chloride in the presence of N,N-dimethylaniline at reflux to produce a reaction mass. The cooled reaction mass is poured onto ice followed by extracting with diethyl ether to afford a solution; then the combined extracts are dried and concentrated. The residue is chromatographed (SiO2, light petrol) to produce 4,6-dichloro-5-nitro-2-(propylthio)pyrimidine.

According to U.S. Pat. No. 6,525,060, ticagrelor is prepared by the condensation of 4,6-dichloro-5-nitro-2-(propylthio)pyrimidine with [3aR-(3aα,4α,6α,6aα)]-6-amino-tetrahydro-2,2-dimethyl-4H-cyclopenta-1,3-dioxol-4-ol, hydrochloride salt in the presence of N,N-diisopropylethylamine in tetrahydrofuran to produce [3aR-(3aα,4α,6α,6aα)]-6-[[6-chloro-5-nitro-2-(propylthio)-pyrimidin-4-yl]amino]-tetrahydro-2,2-dimethyl-4H-cyclopenta-1,3-dioxol-4-ol, followed by reduction in the presence of iron powder in acetic acid to produce [3aR-(3aα,4α,6α,6aα)]-6-[[5-amino-6-chloro-2-(propylthio)-pyrimidin-4-yl]amino]-tetrahydro-2,2-dimethyl-4H-cyclopenta-1,3-dioxol-4-ol, which is then reacted with isoamyl nitrite in acetonitrile to produce [3aR-(3aα,4α,6α,6aα)]-6-[7-chloro-5-(propylthio)-3H-1,2,3-triazolo[4,5-d]-pyrimidin-3-yl]tetrahydro-2,2-dimethyl-4H-cyclopenta-1,3-dioxol-4-ol.

The resulting triazolo[4,5-d]-pyrimidin compound is reacted with ammonia in tetrahydrofuran to produce [3aR-(3aα,4α,6α,6aα)]-6-[7-amino-5-(propylthio)-3H-1,2,3-triazolo[4,5-d]-pyrimidin-3-yl]tetrahydro-2,2-dimethyl-4H-cyclopenta-1,3-dioxol-4-ol, which is then reacted with a solution of trifluoromethanesulfonyloxy-acetic acid methyl ester in tetrahydrofuran in the presence of butyllithium to produce [3aR-(3aα,4α,6α,6aα)]-6-[[7-amino-5-(propylthio)-3H-1,2,3-triazolo[4,5-d]-pyrimidin-3-yl]tetrahydro-2,2-dimethyl-4H-cyclopenta-1,3-dioxol-4-ol]oxy]acetic acid methyl ester, followed by bromination in the presence of isoamylnitrite in bromoform to produce [3aR-(3aα,4α,6α,6aα)]-6-[[7-bromo-5-(propylthio)-3H-1,2,3-triazolo[4,5-d]-pyrimidin-3-yl]tetrahydro-2,2-dimethyl-4H-cyclopenta-1,3-dioxol-4-ol]oxy]acetic acid methyl ester.

The resulting bromo compound is then reacted with (1R-trans)-2-(3,4-difluorophenyl)cyclopropanamine [R—(R*,R*)]-2,3-dihydroxybutanedioate (1:1) salt in the presence of N,N-diisopropylethylamine in dichloromethane to produce [3aR-[3aα,4α,6α(1R*,2S*),6aα]]-[[6-[7-[[2-(3,4-difluorophenyl)cyclopropyl]amino]-5-(propylthio)-3H-1,2,3-triazolo[4,5-d]-pyrimidin-3-yl]tetrahydro-2,2-dimethyl-4H-cyclopenta-1,3-dioxol-4-ol]oxy]acetic acid methyl ester, followed by reaction with DIBAL-H in tetrahydrofuran to produce [3aR-[3aα,4α,6α(1R*,2S*),6aα]]-[[6-[7-[[2-(3,4-difluorophenyl)cyclopropyl]amino]-5-(propylthio)-3H-1,2,3-triazolo[4,5-d]-pyrimidin-3-yl]tetrahydro-2,2-dimethyl-4H-cyclopenta-1,3-dioxol-4-ol]oxy]-ethanol, which is then treated with trifluoroacetic acid in water to produce [1S-(1α,2α,3β(1S*,2R*),5β)]-3-[7-[2-(3,4-difluorophenyl)cyclopropyl]amino]-5-(propylthio)-3H-1,2,3-triazolo[4,5-d]pyrimidin-3-yl)-5-(2-hydroxyethoxy)-cyclopentane-1,2-diol (ticagrelor).

U.S. Patent Application No. 2007/0293513 (hereinafter referred to as the '513 application) discloses four crystalline forms (polymorphs I, II, III and IV) of the compound of formula I (ticagrelor), processes for their preparation, and characterizes the polymorphs by powder X-ray diffraction (P-XRD) pattern and melting points which were determined using differential scanning calorimetry (DSC).

The '513 application teaches an amorphous form of ticagrelor (Form a), and a process for preparing it. According to the teachings of the '513 application, the ticagrelor in substantially amorphous form is produced by a process which comprises freeze drying or spray drying a solution of ticagrelor using a suitable solvent system, for example ethanol/water. As per the process exemplified in the '513 application, the Form a of ticagrelor is prepared by dissolving ticagrelor in a 50% aqueous solution of ethanol, followed by the drop-wise addition of water and then freeze drying the resulting saturated solution using Virtis instrumentation under the following conditions (vacuum 2170 mT, run time 20.2 hours, condensed temperature −52° C., ambient temperature 20.3° C.).

There is a strong technical and commercial desire to develop a modified process for the preparation of novel crystalline form of ticagrelor in order to overcome the problems associated with the prior art processes.

### SUMMARY OF THE INVENTION

The present inventors have now surprisingly and unexpectedly found a novel crystalline form of ticagrelor.

The novel crystalline form of ticagrelor is consistently reproducible, does not have the tendency to convert to other forms, and is found to be more stable. The novel crystalline form disclosed herein exhibits properties making it suitable for formulating ticagrelor.

One aspect, encompassed herein is a process for preparing the novel crystalline form of ticagrelor.

In another aspect, provided herein is a pharmaceutical composition comprising said novel crystalline form of ticagrelor which is preferably essentially free of crystalline forms as disclosed herein and one or more pharmaceutically acceptable excipients.

In still another aspect, provided herein is a pharmaceutical composition comprising the novel crystalline form of ticagrelor which is preferably essentially free of crystalline forms made by the process disclosed herein, and one or more pharmaceutically acceptable excipients.

In still a further aspect, encompassed herein is a process for preparing a pharmaceutical formulation comprising combining said novel crystalline form of ticagrelor which is preferably essentially free of crystalline forms with one or more pharmaceutically acceptable excipients.

The novel crystalline form of ticagrelor of crystalline forms disclosed herein for use in the pharmaceutical compositions can have a D₉₀ particle size of less than or equal to about 400 microns, specifically about 1 micron to about 300 microns, and most specifically about 10 microns to about 150 microns.

### BRIEF DESCRIPTION OF THE DRAWINGS

FIG. 1 illustrates characteristic powder X-ray diffraction (XRD) pattern of the novel crystalline form of ticagrelor

FIG. 2 illustrates Differential scanning calorimetric (DSC) of thermogram novel crystalline form of ticagrelor

### DETAILED DESCRIPTION OF THE INVENTION

Unless otherwise indicated, the following definitions are set forth to illustrate and define the meaning and scope of the various terms used to describe the invention herein.

The term “pharmaceutically acceptable” means that which is useful in preparing a pharmaceutical composition that is generally non-toxic and is not biologically undesirable and includes that which is acceptable for veterinary use and/or human pharmaceutical use.

The term “pharmaceutical composition” is intended to encompass a drug product including the active ingredient(s), pharmaceutically acceptable excipients that make up the carrier, as well as any product which results, directly or indirectly, from combination, complexation or aggregation of any two or more of the ingredients. Accordingly, the pharmaceutical compositions encompass any composition made by admixing the active ingredient, active ingredient dispersion or composite, additional active ingredient(s), and pharmaceutically acceptable excipients.

The term “therapeutically effective amount” as used herein means the amount of a compound that, when administered to a mammal for treating a state, disorder or condition, is sufficient to effect such treatment. The “therapeutically effective amount” will vary depending on the compound, the disease and its severity and the age, weight, physical condition and responsiveness of the mammal to be treated.

The term “delivering” as used herein means providing a therapeutically effective amount of an active ingredient to a particular location within a host causing a therapeutically effective blood concentration of the active ingredient at the particular location. This can be accomplished, e.g., by topical, local or by systemic administration of the active ingredient to the host.

The term “buffering agent” as used herein is intended to mean a compound used to resist a change in pH upon dilution or addition of acid of alkali. Such compounds include, by way of example and without limitation, potassium metaphosphate, potassium phosphate, monobasic sodium acetate and sodium citrate anhydrous and dehydrate and other such material known to those of ordinary skill in the art.

The term “sweetening agent” as used herein is intended to mean a compound used to impart sweetness to a formulation. Such compounds include, by way of example and without limitation, aspartame, dextrose, glycerin, mannitol, saccharin sodium, sorbitol, sucrose, fructose and other such materials known to those of ordinary skill in the art.

The term “binders” as used herein is intended to mean substances used to cause adhesion of powder particles in granulations. Such compounds include, by way of example and without limitation, acacia, alginic acid, tragacanth, carboxymethylcellulose sodium, polyvinylpyrrolidone, compressible sugar (e.g., NuTab), ethylcellulose, gelatin, liquid glucose, methylcellulose, pregelatinized starch, starch, polyethylene glycol, guar gum, polysaccharide, bentonites, sugars, invert sugars, poloxamers (PLURONIC™ F68, PLURONIC™ F127), collagen, albumin, celluloses in non-aqueous solvents, polypropylene glycol, polyoxyethylene-polypropylene copolymer, polyethylene ester, polyethylene sorbitan ester, polyethylene oxide, microcrystalline cellulose, combinations thereof and other material known to those of ordinary skill in the art.

The term “diluent” or “filler” as used herein is intended to mean inert substances used as fillers to create the desired bulk, flow properties, and compression characteristics in the preparation of solid dosage formulations. Such compounds include, by way of example and without limitation, dibasic calcium phosphate, kaolin, sucrose, mannitol, microcrystalline cellulose, powdered cellulose, precipitated calcium carbonate, sorbitol, starch, combinations thereof and other such materials known to those of ordinary skill in the art.

The term “glidant” as used herein is intended to mean agents used in solid dosage formulations to improve flow-properties during tablet compression and to produce an anti-caking effect. Such compounds include, by way of example and without limitation, colloidal silica, calcium silicate, magnesium silicate, silicon hydrogel, cornstarch, talc, combinations thereof and other such materials known to those of ordinary skill in the art.

The term “lubricant” as used herein is intended to mean substances used in solid dosage formulations to reduce friction during compression of the solid dosage.

Such compounds include, by way of example and without limitation, calcium stearate, magnesium stearate, mineral oil, stearic acid, zinc stearate, combinations thereof and other such materials known to those of ordinary skill in the art.

The term “disintegrant” as used herein is intended to mean a compound used in solid dosage formulations to promote the disruption of the solid mass into smaller particles which are more readily dispersed or dissolved. Exemplary disintegrants include, by way of example and without limitation, starches such as corn starch, potato starch, pregelatinized, sweeteners, clays, such as bentonite, microcrystalline cellulose (e.g., Avicel™), carsium (e.g., Amberlite™), alginates, sodium starch glycolate, gums such as agar, guar, locust bean, karaya, pectin, tragacanth, combinations thereof and other such materials known to those of ordinary skill in the art.

The term “wetting agent” as used herein is intended to mean a compound used to aid in attaining intimate contact between solid particles and liquids. Exemplary wetting agents include, by way of example and without limitation, gelatin, casein, lecithin (phosphatides), gum acacia, cholesterol, tragacanth, stearic acid, benzalkonium chloride, calcium stearate, glycerol monostearate, cetostearyl alcohol, cetomacrogol emulsifying wax, sorbitan esters, polyoxyethylene alkyl ethers (e.g., macrogol ethers such as cetomacrogol 1000), polyoxyethylene castor oil derivatives, polyoxyethylene sorbitan fatty acid esters, (e.g., TWEEN™s), polyethylene glycols, polyoxyethylene stearates colloidal silicon dioxide, phosphates, sodium dodecylsulfate, carboxymethylcellulose calcium, carboxymethylcellulose sodium, methylcellulose, hydroxyethylcellulose, hydroxyl propylcellulose, hydroxypropylmethylcellulose phthalate, noncrystalline cellulose, magnesium aluminum silicate, triethanolamine, polyvinyl alcohol, and polyvinylpyrrolidone (PVP).

The term “micronization” used herein means a process or method by which the size of a population of particles is reduced.

As used herein, the term “micron” or “μm” both are same refers to “micrometer” which is 1×10⁻⁶ meter.

As used herein, “crystalline particles” means any combination of single crystals, aggregates and agglomerates.

As used herein, “Particle Size Distribution (PSD)” means the cumulative volume size distribution of equivalent spherical diameters as determined by laser diffraction in Malvern Master Sizer 2000 equipment or its equivalent. “Mean particle size distribution, i.e., (D₅₀)” correspondingly, means the median of said particle size distribution.

The important characteristics of the PSD are the (D₉₀), which is the size, in microns, below which 90% of the particles by volume are found, and the (D₅₀), which is the size, in microns, below which 50% of the particles by volume are found. Thus, a D₉₀ or d(0.9) of less than 300 microns means that 90 volume-percent of the particles in a composition have a diameter less than 300 microns.

The term “highly pure” is meant having total purity, which includes both chemical and enantiomeric purity, greater than about 99%, specifically greater than about 99.5%, and more specifically greater than about 99.9% measured by HPLC.

As used herein, “reflux temperature”/“boiling point means” the temperature at which the solvent or solvent system refluxes or boils at atmospheric pressure.

The present invention relates to the following items:

(1) Crystalline form of ticagrelor of Formula I:

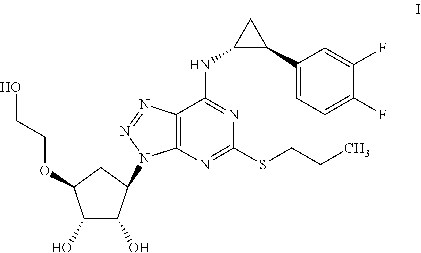 [SMILES: CCCSc1nc(N[C@@H]2C[C@H]2c2ccc(F)c(F)c2)c2nnn([C@@H]3C[C@H](OCCO)[C@@H](O)[C@H]3O)c2n1]

characterized by an X-ray powder diffraction pattern having peaks at 4.78±0.2, 5.97±0.2, 6.91±0.2, 8.25±0.2, 9.49±0.2, 11.95±0.2, 13.76±0.2, 14.31±0.2, 16.42±0.2, 16.75±0.2, 18.99±0.2, 20.13±0.2, 20.41±0.2, 20.67±0.2, 23.21±0.2, 24.34±0.2, and 29.67±0.2 degrees 2-theta, when using Copper-anode wavelength of λ=1.5406 Angstrom.

In particular, the crystalline form of ticagrelor according to the present invention is free of polymorphs I-IV of US 2007/0293513 A1, which are described in the following:

Polymorph I, when it is substantially pure and essentially in the anhydrous form, has an X-ray powder diffraction pattern containing specific peaks at 5.3±0.1, 8.0±0.1, 9.6±0.1, 13.9±0.1, 15.3±0.1, 20.1±0.1, 20.7±0.1, 21.0±0.1, 21.3±0.1, 26.2±0.1 and 27.5±0.1 degrees 2-theta.

Polymorph II, when it is substantially pure and essentially in the anhydrous form, has an X-ray powder diffraction pattern containing specific peaks at 5.5±0.1, 6.8±0.1, 10.6±0.1, 13.5±0.1, 14.9±0.1, 18.3±0.1, 19.2±0.1, 22.7±0.1, 24.3±0.1, and 27.1±0.1 degrees 2-theta.

Polymorph III, when it is substantially pure and essentially in the anhydrous form, has an X-ray powder diffraction pattern containing specific peaks at 5.6±0.1, 12.5±0.1, 14.0±0.1, 17.4±0.1, 18.4±0.1, 21.4±0.1, 22.2±0.1, 22.9±0.1, 24.1±0.1, and 24.5±0.1 degrees 2-theta.

Polymorph IV, when it is substantially pure and essentially in the anhydrous form, has an X-ray powder diffraction pattern containing specific peaks at 4.9±0.1, 6.0±0.1, 9.2±0.1, 11.6±0.1, 12.80±0.1, 15.6±0.1, 16.4±0.1, 17.2±0.1, and 18.1±0.1 degrees 2-theta.

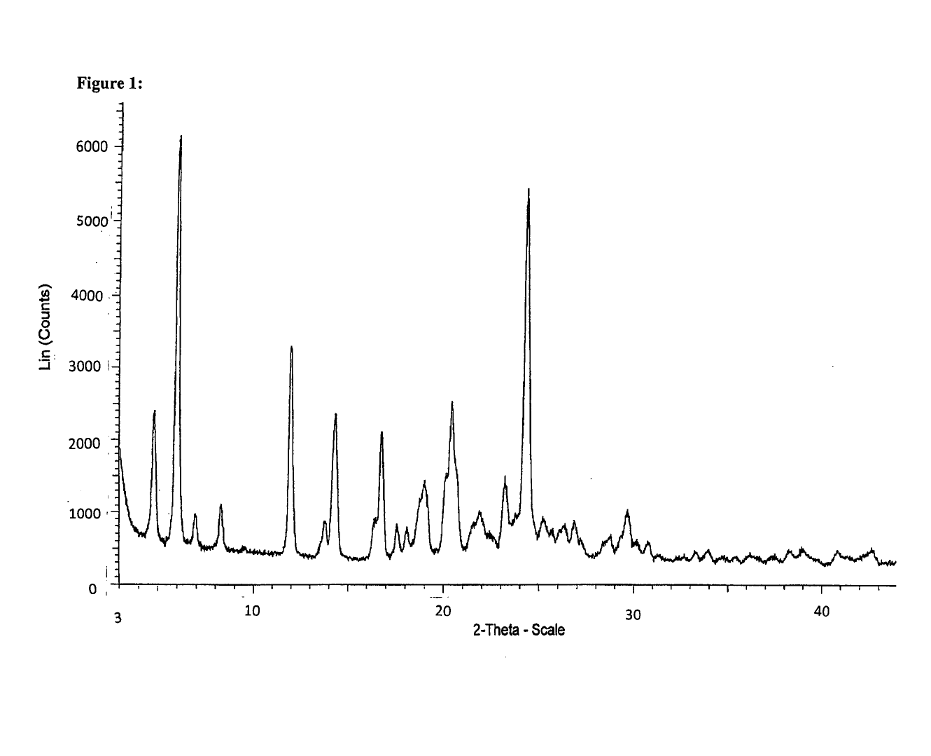

(2) The crystalline form of ticagrelor of item (1), wherein the chemical and/or chiral purity of the crystalline form is greater than 99%, specifically greater than 99.5%, as measured by HPLC.
(3) The crystalline form of ticagrelor of item (1) or (2), wherein said crystalline form is essentially free of other crystalline forms.
(4) The crystalline form of ticagrelor of any of the preceding items, wherein said crystalline form is essentially free of other crystalline forms of ticagrelor that is to say that no other crystalline forms of ticagrelor can be detected by X-ray powder diffraction measurement when using Copper-anode wavelength of ?=1.5406 Angstrom. Preferably, the X-ray powder diffraction pattern of the crystalline form of ticagrelor is free of any other compounds in crystalline form, meaning that only peaks of the crystalline form of ticagrelor according to the invention (see also FIG. 1) can be detected.
(5) The crystalline form of ticagrelor of any of the preceding items, having a D₉₀ particle size of less than or equal to 400 microns, specifically 1 micron to 300 microns, as determined by laser diffraction.
(6) Process for the preparation of the crystalline form of ticagrelor according to items 1-5, comprising the steps of:
a) providing a first solution or a suspension of ticagrelor in a first polar solvent;
b) optionally heating said suspension to get a clear first solution;
c) adding said first solution into a second ether solvent at a temperature in the range of from −70° C. to below 5° C., or adding a second ether solvent into said first solution at a temperature of in the range of from −70° C. to below 5° C.;
d) adding a third alcohol or ester solvent; and
e) recovering said crystalline form of ticagrelor.
   (7) The process of item 6, wherein the first polar solvent in step (a) is selected from the group consisting of an amide solvent, a mixture of amide solvents, dimethyl sulphoxide, and mixtures of the aforementioned; preferably the first polar solvent is selected from the group consisting of dimethyl formamide, dimethyl acetamide, dimethyl sulphoxide, and mixtures of the aforementioned; preferably, the first polar solvent is N,N-dimethyl formamide. The first polar solvent may also be a solvent that essentially consists of polar solvents, meaning that small amounts of non-polar solvents, such as up to 10% by volume of non-polar solvents, may also be present. Likewise, if the first polar solvent includes the preferred solvents defined above, small amounts of other solvents, such as up to 10% by volume of other solvents may also be present.
   (8) The process of item 6 or 7, wherein heating said suspension in step (b) is performed at a temperature in the range of from 45° C. to 80° C., and preferably below the boiling point of the suspension. “Below the boiling point of the suspension” may be 1° C., 5° C., or 10° C. below the boiling point of said suspension. The suspension can be heated until a visually clear solution is formed.
   (9) The process of any of items 6-8, wherein the second ether solvent is selected from the group consisting of tetrahydrofuran, isopropyl ether, diisopropyl ether, diethyl ether and mixtures thereof; preferably the second ether solvent is diisopropyl ether. The second ether solvent may also be a solvent that essentially consists of ether solvents, meaning that small amounts of non-ether solvents, such as up to 10% by volume of non-ether solvents, may also be present. Likewise, if the second ether solvent includes the preferred ether solvents defined above, small amounts of other solvents, such as up to 10% by volume of other solvents may also be present.
   (10) The process of any of items 6-9, wherein the second ether solvent in step (c) is used in 10 to 50 volumes based on the first polar solvent.
   (11) The process of any of items 6-10, wherein the third alcohol or ester solvent is selected from the group consisting of methanol, ethanol, isopropyl alcohol, ethyl acetate, and mixtures of the aforementioned; preferably the third ester solvent is ethyl acetate. The third alcohol or ester solvent may also be a solvent that essentially consists of alcohol and/or ester solvents, meaning that small amounts of non-alcohol or non-ester solvents, such as up to 10% by volume, may also be present. Likewise, if the third alcohol or ester solvent includes the preferred solvents defined above, small amounts of other solvents, such as up to 10% by volume of other solvents may also be present. Preferably, the third alcohol or ester solvent in step (c) is used in 0.5 to 5 volumes based on the first polar solvent.
   (12) The process of any of items 6-11, wherein the addition of the third alcohol or ester solvent in step (d) takes place under stirring at a temperature in the range of from −20° C. to 20° C. or from −10° C. to 10° C. Preferably, the addition of the third alcohol or ester solvent in step (d) takes place at a temperature of about 0° C. As referred to herein, adding one solvent to another solvent at a specific temperature range means that the temperature of one solvent is set to a temperature within said range and the other solvent, which shall be added, is slowly added to said one solvent so that the temperature can be maintained in the desired range. The addition of solvents according to the present invention can be performed under nitrogen atmosphere.
   (13) Crystalline form of ticagrelor of Formula I:

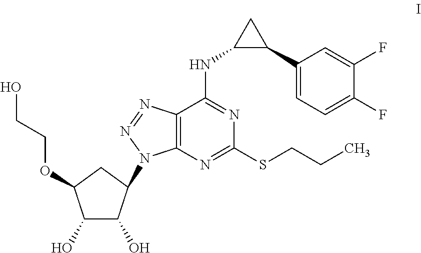 [SMILES: CCCSc1nc(N[C@@H]2C[C@H]2c2ccc(F)c(F)c2)c2nnn([C@@H]3C[C@H](OCCO)[C@@H](O)[C@H]3O)c2n1]

obtainable or obtained by using a process as defined in any of items 6-12.

(14) Pharmaceutical composition, specifically a solid dosage form or oral suspension, comprising the crystalline form of ticagrelor of Formula I according to any of items 1-5 and 13 and one or more pharmaceutically acceptable excipients, preferably wherein the pharmaceutical composition is essentially free of further crystalline forms so that no other crystalline forms of ticagrelor can be detected by X-ray powder diffraction measurement when using Copper-anode wavelength of λ=1.5406 Angstrom.

(15) Crystalline form of ticagrelor according to any of items 1-5 and 13 or the pharmaceutical composition of item 14 for use as medicament, preferably for use in the prevention of cardiovascular events including myocardial infarction, stroke, and cardiovascular death in patients with acute coronary syndrome or for use in the treatment of thrombosis, angina, ischemic heart diseases and coronary artery diseases.
(16) Process for preparing a pharmaceutical composition according to item 14, wherein the process comprises combining the crystalline form of ticagrelor according to any of items 1-5 and 13 with one or more pharmaceutically acceptable excipients
The above items are described in more detail below.

According to one aspect, there is provided a novel crystalline form of ticagrelor which is preferably a stable crystalline form of ticagrelor essentially free of other crystalline forms.

The novel crystalline form of ticagrelor which is preferably essentially free of other crystalline forms is characterized by a powder XRD pattern substantially in accordance with FIG. 1.

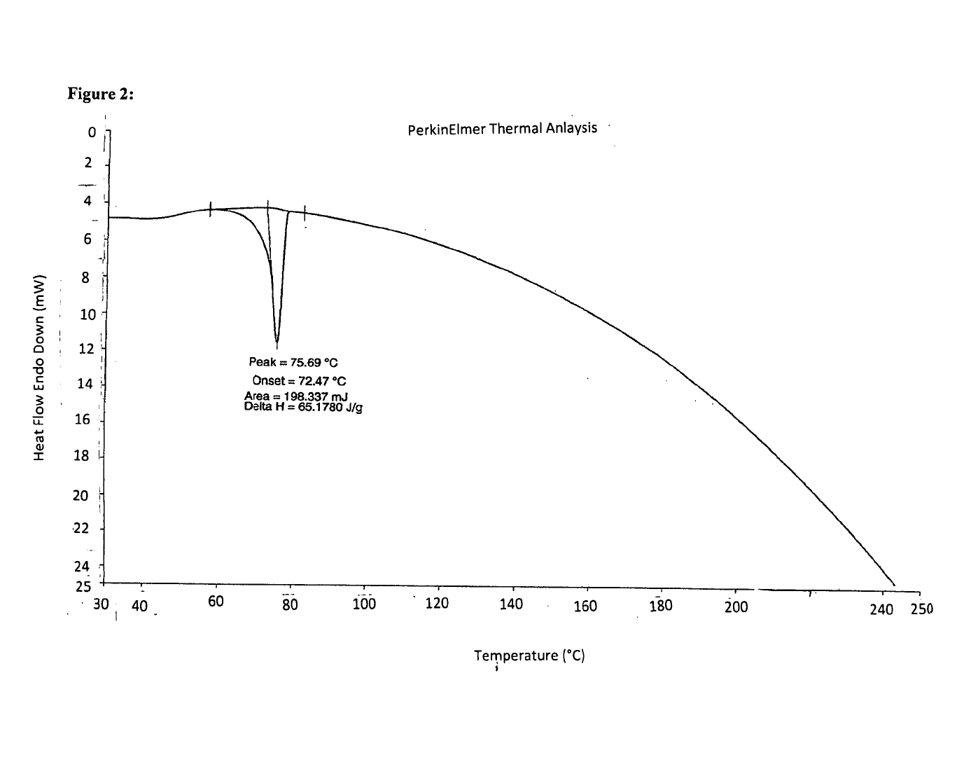

The crystalline form of ticagrelor is characterized by one or more of the following properties:
i) a powder X-ray diffraction pattern substantially in accordance with FIG. 1;
ii) a powder X-ray diffraction pattern having peaks at about and 4.78, 5.97, 6.91, 8.25, 9.49, 11.95, 13.76, 14.31, 16.42, 16.75, 18.99, 20.13, 20.41, 20.67, 23.21, 24.34, and 29.67±0.2 degrees 2-theta;
iii) DSC thermogram substantially in accordance FIG. 2;

According to another aspect, there is provided a process for the preparation of novel crystalline form of ticagrelor essentially free of crystalline forms, comprising:

a) providing a solution of ticagrelor in an first solvent;
b) optionally, heating the suspension to get clear solution;
c) adding first solution in to second solvent at lower temperature;
d) adding third solvent and
e) recovering novel crystalline form of ticagrelor.

The novel crystalline form of ticagrelor which is essentially free of other crystalline forms obtained by the process disclosed herein is stable, consistently reproducible, has good dissolution properties, and is particularly suitable for bulk preparation and handling. The novel crystalline form of ticagrelor essentially free of other crystalline forms obtained by the process disclosed herein is suitable for formulating ticagrelor.

Step-(a) of providing a solution of ticagrelor includes dissolving ticagrelor in the polar solvent such as amide solvent, or obtaining an existing solution from a previous processing step. The suitable amide solvents include, but are not limited to, dimethyl formamide, dimethyl acetamide and non amide solvents like dimethyl sulphoxide.

Ticagrelor can be dissolved in the solvent at a temperature of below about reflux temperature of the solvent used, specifically at about 45° C. to about 80° C., and still more specifically at about 60° C. to about 65° C.

Step-(c) can include the addition of solution of step (b) into second solvent such as ether solvent at lower temperature. The suitable ether solvent include, but are not limited to, tetrahydrofuran, isopropyl ether, diisopropyl ether, diethyl ether and mixture thereof. The solvent can be used in about 10 to 50 volumes and preferably in about 30-40 volumes. Step-(c) can also include the addition of the second ether solvent into the solution of step (b) at lower temperature. The suitable ether solvent include, but are not limited to, tetrahydrofuran, isopropyl ether, diisopropyl ether, diethyl ether and mixture thereof. The solvent can be used in about 10 to 50 volumes and preferably in about 30-40 volumes.

The second solvent addition can take place at a temperature of below 5° C., specifically at about 0° C. to about −70° C., and still more specifically at about 0° C. to about 5° C.

Step-(d) includes addition of third solvent such as alcohol like methanol, ethanol, isopropyl alcohol, etc., esters like ethyl acetate, etc. The addition of third solvent preferably takes place under stirring at a temperature about 0° C.

After completion of addition process, the resulting mass is preferably stirred at a temperature of temperature of about 15° C. to about 45° C. from about 1 hour to 2 hours, for at least 1 hour and more preferably at a temperature of about 25° C. to about 30° C. from about 1 hour.

Step-(e) includes recovering of novel crystalline form of ticagrelor by collecting resulted solid from the reaction mixture by various technique known in the art.

The novel crystalline form of ticagrelor obtained by above process may be further dried in, for example, Vacuum Tray Dryer, Rotocon Vacuum Dryer, Vacuum Paddle Dryer or pilot plant Rota vapor, to further lower residual solvents. Drying can be carried out under reduced pressure until the residual solvent content reduces to the desired amount such as an amount that is within the limits given by the International Conference on Harmonization of Technical Requirements for Registration of Pharmaceuticals for Human Use (“ICH”) guidelines. Drying can be suitably carried out in a tray dryer, vacuum oven, air oven, or using a fluidized bed drier, spin flash dryer, flash dryer, and the like. Drying equipment selection is well within the ordinary skill in the art.

The total purity, including the chemical and/or chiral purity, of the novel crystalline form of ticagrelor obtained by the process disclosed herein is preferably greater than about 99%, specifically greater than about 99.5%, more specifically greater than about 99.9%, and most specifically greater than about 99.95% as measured by HPLC.

Ticagrelor as used herein as starting material can be obtained by processes described in the prior art, for example by the process described in the U.S. Pat. Nos. 6,251,910 and 6,525,060.

Further encompassed herein is the use of the novel crystalline form of ticagrelor for the manufacture of a pharmaceutical composition together with a pharmaceutically acceptable carrier.

A specific pharmaceutical composition of novel crystalline form of ticagrelor is selected from a solid dosage form and an oral suspension.

The novel crystalline form of ticagrelor crystalline forms can have a D₉₀ particle size of less than or equal to about 400 microns, specifically about 1 micron to about 300 micron, and most specifically about 10 microns to about 150 microns.

The particle sizes of novel crystalline form of ticagrelor can be produced by a mechanical process of reducing the size of particles which includes any one or more of cutting, chipping, crushing, milling, grinding, micronizing, trituration or other particle size reduction methods known in the art, to bring the solid state form to the desired particle size range.

According to another aspect, there is provided a method for treating a patient suffering from thrombosis, angina, Ischemic heart diseases and coronary artery diseases, comprising administering a therapeutically effective amount of the novel crystalline form of ticagrelor, or a pharmaceutical composition that comprises a therapeutically effective amount of highly pure amorphous ticagrelor essentially free of crystalline forms along with pharmaceutically acceptable excipients.

According to another aspect, there is provided pharmaceutical compositions comprising novel crystalline form of ticagrelor prepared according to the processes disclosed herein and one or more pharmaceutically acceptable excipients.

Pharmaceutical compositions comprise at least a therapeutically effective amount of the novel crystalline form of ticagrelor. Such pharmaceutical compositions may be administered to a mammalian patient in a dosage form, e.g., solid, liquid, powder, elixir, aerosol, syrup, injectable solution, etc. Dosage forms may be adapted for administration to the patient by oral, buccal, parenteral, ophthalmic, rectal and transdermal routes or any other acceptable route of administration. Oral dosage forms include, but are not limited to, tablets, pills, capsules, syrup, troches, sachets, suspensions, powders, lozenges, elixirs and the like. The novel crystalline form of ticagrelor may also be administered as suppositories, ophthalmic ointments and suspensions, and parenteral suspensions, which are administered by other routes.

The pharmaceutical compositions further contain one or more pharmaceutically acceptable excipients. Suitable excipients and the amounts to use may be readily determined by the formulation scientist based upon experience and consideration of standard procedures and reference works in the field, e.g., the buffering agents, sweetening agents, binders, diluents, fillers, lubricants, wetting agents and disintegrants described hereinabove.

Capsule dosage forms contain novel crystalline form of ticagrelor within a capsule which may be coated with gelatin. Tablets and powders may also be coated with an enteric coating. Suitable enteric coating agents include phthalic acid cellulose acetate, hydroxypropylmethyl cellulose phthalate, polyvinyl alcohol phthalate, carboxy methyl ethyl cellulose, a copolymer of styrene and maleic acid, a copolymer of methacrylic acid and methyl methacrylate, and like materials, and if desired, the coating agents may be employed with suitable plasticizers and/or extending agents. A coated capsule or tablet may have a coating on the surface thereof or may be a capsule or tablet comprising a powder or granules with an enteric-coating.

Tableting compositions may have few or many components depending upon the tableting method used, the release rate desired and other factors. For example, the compositions described herein may contain diluents such as cellulose-derived materials like powdered cellulose, microcrystalline cellulose, microfine cellulose, methyl cellulose, ethyl cellulose, hydroxyethyl cellulose, hydroxypropyl cellulose, hydroxypropylmethyl cellulose, carboxymethyl cellulose salts and other substituted and unsubstituted celluloses; starch; pregelatinized starch; inorganic diluents such calcium carbonate and calcium diphosphate and other diluents known to one of ordinary skill in the art. Yet other suitable diluents include waxes, sugars (e.g. lactose) and sugar alcohols such as mannitol and sorbitol, acrylate polymers and copolymers, as well as pectin, dextrin and gelatin.

Other excipients include binders, such as acacia gum, pregelatinized starch, sodium alginate, glucose and other binders used in wet and dry granulation and direct compression tableting processes; disintegrants such as sodium starch glycolate, crospovidone, low-substituted hydroxypropyl cellulose and others; lubricants like magnesium and calcium stearate and sodium stearyl fumarate; flavorings; sweeteners; preservatives; pharmaceutically acceptable dyes and glidants such as silicon dioxide.

### Instrumental Details:

### X-Ray Powder Diffraction (P-XRD):

The X-Ray powder diffraction pattern was obtained by a Bruker AXS,D8 Advance X-ray powder diffractometer in Theta-Theta configuration using Copper-anode wavelength (λ=1.5406 Angstrom) operated at 40 KV and 40 mA. The sample was analyzed in the scan range 3-45° 2θ; step width=0.01579°; and measuring time per step=0.11 second.

### Differential Scanning Calorimetric (DSC) of Thermogram

The Differential Scanning calorimetry thermogram was obtained using Perkin Elmer Diamond DSC instrument. The pan type was pierced aluminum pan. The analysis was carried out under a flow of nitrogen gas (20 ml/min), and the Temperature range 30° C. to 250° C. at a constant rate of temperature increase of 10° C./min.

The following examples are provided to enable one skilled in the art to practice the invention and are merely illustrate the process of this invention. However, it is not intended in any way to limit the scope of the present invention.

### EXAMPLES

### Example 1

### Preparation of Novel Crystalline Form of Ticagrelor

[1S-[1a,2a,3b(1S*,2R*),5b]]-3-[7-[2-[3,4-difluorophenyl)-cyclopropylamino]-5-(propylthio)-3H-1,2,3-triazolo[4,5-d]pyrimidin-3-yl]-5-(2-hydroxyethoxyl)cyclopentane-1,2-diol (10 gm) was dissolved in N,N-dimethylformamide (12 ml) at 60-65° C. to get clear solution. This was followed by addition of diisopropyl ether (225 ml) at −70° C. drop wise under stirring and under nitrogen atmosphere. The temperature of suspension was raised up to 0° C. Ethyl acetate (20 ml) was added under stirring at 0° C. The temperature of suspension was raised to 25-30° C. The resulted suspension was further stirred at 25-30° C. for 1 hour. The resulted solid was filtered to get ticagrelor (8 gm).

### Example 2

### Preparation of Novel Crystalline Form of Ticagrelor

[1S-[1a,2a,3b(1S*,2R*),5b]]-3-[7-[2-(3,4-difluorophenyl)-cyclopropylamino]-5-(propylthio)-3H-1,2,3-triazolo[4,5-d]pyrimidin-3-yl]-5-(2-hydroxyethoxyl)cyclopentane-1,2-diol (10 gm) was dissolved in N,N-dimethylformamide (12 ml) at 60-65° C. to get clear solution. This was followed by addition of diisopropyl ether (225 ml) at 0° C. to 5° drop wise under stirring and under nitrogen atmosphere. The temperature of suspension was raised up to 0° C. Ethyl acetate (20 ml) was added under stirring at 0° C. The temperature of suspension was raised to 25-30° C. The resulted suspension was further stirred at 25-30° C. for 1 hour. The resulted solid was filtered to get ticagrelor (8 gm).

### Analytical Result:

The ticagrelor solid obtained by the above process is characterized by an X-ray powder diffraction pattern as depicted in FIG. 1.

### CITED DOCUMENTS

1 EP0996621 A1 (published as WO 9905143 A1);
2 EP1135391 A1 (published as WO 00/34283 A1);
3 U.S. Pat. No. 5,747,496;
4 U.S. Pat. No. 6,251,910 B1;
5 U.S. Pat. No. 6,525,060 B1;
6 U.S. Pat. No. 6,974,868 B2;
7 U.S. Pat. No. 7,067,663 B2;
8 U.S. Pat. No. 7,250,419 B2;

### 9 US 2007/0265282 A1;

### 10 US 2007/0293513 A1;

### 11 US 2008/0214812 A1;

### 12 WO2008/018823; and

13 WO2010/030224 A1.

1. A crystalline form of ticagrelor of Formula I: 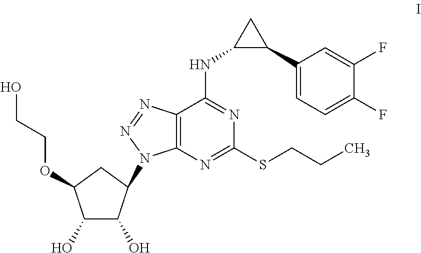 [SMILES: CCCSc1nc(N[C@@H]2C[C@H]2c2ccc(F)c(F)c2)c2nnn([C@@H]3C[C@H](OCCO)[C@@H](O)[C@H]3O)c2n1]
characterized by an X-ray powder diffraction pattern having peaks at 4.78±0.2, 5.97±0.2, 6.91±0.2, 8.25±0.2, 9.49±0.2, 11.95±0.2, 13.76±0.2, 14.31±0.2, 16.42±0.2, 16.75±0.2, 18.99±0.2, 20.13±0.2, 20.41±0.2, 20.67±0.2, 23.21±0.2, 24.34±0.2, and 29.67±0.2 degrees 2-theta,
when using Copperanode wavelength of λ=1.5406 Angstrom.

2. The crystalline form of ticagrelor of claim 1, wherein the chemical and/or chiral purity of the crystalline form is greater than 99%.

3. The crystalline form of ticagrelor of claim 1, wherein said crystalline form is essentially free of other crystalline forms.

4. The crystalline form of ticagrelor of claim 3, wherein said crystalline form is such that no other crystalline forms of ticagrelor are detectable by X-ray powder diffraction measurement when using Copperanode wavelength of λ=1.5406 Angstrom.

5. The crystalline form of ticagrelor of claim 1, having a D₉₀ particle size of less than or equal to 400 microns, specifically 1 micron to 300 microns, as determined by laser diffraction.

6. A process for the preparation of the crystalline form of ticagrelor of claim 1, comprising:
a) providing a first solution or a suspension of ticagrelor in a polar solvent;
b) optionally heating said first solution or suspension to produce a clear first solution;
c) adding said first solution or suspension or clear first solution into an ether solvent at a temperature in the range of from −70° C. to below 5° C., or adding an ether solvent into said first solution or suspension or clear first solution at a temperature of in the range of from −70° C. to below 5° C.;
d) adding an alcohol or ester solvent to the solution of step c); and
e) recovering said crystalline form of ticagrelor from the solution of step d).

7. The process of claim 6, wherein the polar solvent in step (a) is selected from the group consisting of an amide solvent, a mixture of amide solvents, dimethyl sulphoxide, and mixtures of the foregoing polar solvents.

8. The process of claim 6, wherein heating said suspension in step (b) is performed at a temperature in the range of from 45° C. to 80° C.

9. The process of claim 6, wherein the ether solvent is selected from the group consisting of tetrahydrofuran, isopropyl ether, diisopropyl ether, diethyl ether and mixtures thereof.

10. The process of claim 6, wherein the ether solvent in step (c) is used in 10 to 50 volumes based on the volume of the first polar solvent.

11. The process of claim 6, wherein the alcohol or ester solvent is selected from the group consisting of methanol, ethanol, isopropyl alcohol, ethyl acetate, and mixtures thereof.

12. The process of claim 6, wherein the addition of the alcohol or ester solvent in step (d) takes place under stirring at a temperature in the range of from −20° C. to 20° C.

13. A crystalline form of ticagrelor of Formula I: 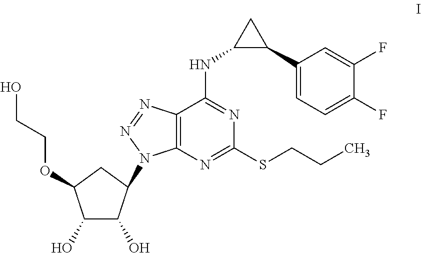 [SMILES: CCCSc1nc(N[C@@H]2C[C@H]2c2ccc(F)c(F)c2)c2nnn([C@@H]3C[C@H](OCCO)[C@@H](O)[C@H]3O)c2n1]
obtainable or obtained by using a process as defined in claim 6.

14. A pharmaceutical composition comprising the crystalline form of ticagrelor of Formula I according to any of claim 1 and one or more pharmaceutically acceptable excipients.

15. A method of treating cardiovascular events including myocardial infarction, stroke, and cardiovascular death in patients with acute coronary syndrome; or for use in the treatment of thrombosis, angina, ischemic heart diseases and coronary artery diseases, by administering to an individual the crystalline form of ticagrelor of claim 1.

16. (canceled)

17. The pharmaceutical composition of claim 14, in the form of a solid dosage form.
